(19) United States
(12) Patent Application Publication
(10) Pub. No.: US 2025/0057487 A1
(43) Pub. Date: Feb. 20, 2025

(54) DEVICES AND METHODS FOR MONITORING PHYSIOLOGICAL PARAMETERS OF PATIENTS AND DISPLAYING CUSTOMIZABLE PARAMETER CONFIGURATIONS

(71) Applicant: Drägerwerk AG & Co. KGaA, Lübeck (DE)

(72) Inventors: Rajesh S. Rane, Andover, MA (US); Devin Weidner, Litchfield, NH (US)

(73) Assignee: Drägerwerk AG & Co. KGaA, Lübeck (DE)

(21) Appl. No.: 18/724,229

(22) PCT Filed: Dec. 22, 2022
(86) PCT No.: PCT/IB2022/062700

§ 371 Date: Jun. 26, 2024

Related U.S. Application Data
(60) Provisional application No. 63/294,976, filed on Dec. 30, 2021.

(51) Int. Cl. A61B 5/00; A61B 5/0205; G16H 10/60; G16H 40/67

(52) U.S. Cl. CPC A61B 5/7425; A61B 5/0205; A61B 5/7435; A61B 5/7475; G16H 10/60; G16H 40/67; A61B 5/681

(57) ABSTRACT

Devices and methods for executing a customizable configuration for displaying one or more physiological parameters of a patient are disclosed. The device includes a display configured to display information related to the patient including physiological data. The device provides a graphical user interface (GUI) on the display including a locked sub-area and a scrollable sub-area, respectively, to display measured values of the physiological parameters. Upon receipt of an input, the device allows the measured values of the physiological parameters to be further configured between the locked sub-area and the scrollable sub-area.

### TECHNICAL FIELD

The present disclosure relates generally to the field of monitoring physiological parameters of patients and displaying customizable parameter configurations. To be specific, the present disclosure relates to displaying patient parameters and waveforms by locking certain areas on display and allowing certain scrollable areas in real-time based on the clinical priority of the parameters or upon users' configurations for visualizing more information that may not be readily visible on display due to limited screen size, and/or for clinical correlation of the parameters between the locked area and scrollable area of the display

### BACKGROUND

A physiological patient monitor displays and records a patient's physiological parameters (e.g., ECG, Temperature, blood pressure, pulse saturation and rate, respiratory rate, anesthesia gas information, brain consciousness levels etc.) associated with the patient's clinical conditions. In reliance on the displayed (and recorded) physiological parameters, clinical providers identify or anticipate the medical conditions of the patient and provide intervention and relevant treatment as clinically needed and suitable. Thus, the display of the physiological parameters, including what clinical information is displayed and how it is displayed, can affect the ability and efficiency of clinical providers to interpret the parameters and take actions thereupon. The physiological patient monitor can come in various forms (e.g., bedside monitor of various sizes, portable/transport small monitor or a combination of portable and expanded larger display, or a remote monitoring handheld device such as a tablet or phone. A small-sized and lightweight portable/transport monitor is suitable in low to medium acuity care areas as a stand-alone monitor. Combination of the portable monitor with an expanded display is suitable for use in higher acuity care areas and for comprehensive visualization of patients' information (e.g., patient monitoring information as well as ventilatory information from a ventilation device or information from a baby warmer or incubator all on one expanded display). The portable monitor is suitable to carry along with the patient for transport to different care area locations for medical treatment. When away from the bedside of the patient, clinical providers also use remote patient monitoring tablets or phones displaying the physiological parameters, to monitor the patient's medical conditions in real-time from a remote location (e.g., physician's office, clinic, or in a remote anesthesia duty room).

One drawback with portable patient monitors and remote monitoring tablets/phones is that they can only display a limited amount of patient data due to the small size of the display even though many more measurements signals are coming into it. By reviewing the data that is displayed on such small monitoring devices, clinical providers only have a limited picture of the patient's medical condition. Frequently, clinical providers have to spend additional time re-configuring the display settings (which may be under authorized protection) to review additional patient data that is not readily apparent on the monitor screen. However, taking this additional time can delay and adversely affect patient treatment, particularly in acute and critical care situations where clinical providers must immediately respond to time-sensitive or life-threatening conditions of the patient. A combination of portable monitor and a bigger expanded display provides fuller information on the expanded display. However, the expanded display is pre-configured and making temporary changes in real-time may not only be inconvenient but more importantly, time consuming.

There exists a need for displaying patient data on portable patient monitoring devices and/or expanded displays in a configurable manner on the fly during real-time monitoring, such that clinical providers can easily navigate, visualize and review patient data quickly and conveniently based on the priority of the patient parameters for clinical correlation with one another, the specific clinical conditions or treatments that the patient is under, and/or other patient-related information (e.g., patient location) as needed. There also exists a need to improve clinical workflow for clinical providers to quickly enable configuring the display layout and review patient data that is not immediately apparent, which will reduce stress and cognitive load on clinical providers, support rapid patient assessment and accurate clinical documentation, and improve the overall patient care.

### SUMMARY

To resolve at least one or more of the above problems and potentially other present or future problems, one aspect of the present disclosure relates to an electronic device capable of executing a customizable configuration in real-time for displaying one or more physiological parameters of a patient. The device includes a display configured to display information related to the patient including physiological data, a memory configured to store one or more programs, and one or more processors configured to execute the one or more programs. The one or more processors provide a graphical user interface (GUI) on the display, where the GUI includes a first sub-area configured to display a measured value or curves of the one or more physiological parameters, and a visual indicator indicating a locked status of the first sub-area of display and a second sub-area configured to display a measured value or curves of at least two or more physiological parameters, and a visual indicator indicating a scrollable status of the second sub-area. Upon receipt of an input, the one or more processors cause the measured value of the one or more physiological parameters to be displayed in one of the first sub-area which is fixed and the second sub-area which is scrollable.

Another aspect of the present disclosure relates to a method of executing a customizable configuration for displaying one or more physiological parameters of a patient on an electronic device. The method includes the step of providing a graphical user interface (GUI) on a display of the electronic device, where the GUI includes a first sub-area configured to display a measured value of the one or more physiological parameters, and a visual indicator indicating a locking status of the first sub-area, and a second sub-area configured to display a measured value of at least two or more other physiological parameters, and a visual indicator indicating a scrollable status of the second sub-area. Upon receipt of an input, the method further includes the step of causing the measured value of the one or more physiological parameters to be displayed in one of the first sub-area and the second sub-area.

### BRIEF DESCRIPTION OF THE DRAWINGS

In the drawings, like reference numbers generally indicate identical, functionally similar, and/or structurally similar elements.

FIG. 1 is a schematic diagram of an exemplary system configured to monitor physiological parameters of a patient and display customizable parameter configurations according to an embodiment of the present disclosure;

FIG. 2 is a schematic diagram of an exemplary device configured to monitor physiological parameters of a patient and displaying customizable parameter configurations according to an embodiment of the present disclosure;

FIG. 3 is a schematic diagram of an exemplary system including a network, a physiological monitoring device, an expanded display which is referred to as multimodality patient-care device hence forth, a remote patient watch device, a central nurse station and a therapy device, according to one embodiment of the present disclosure;

FIG. 4 is a schematic diagram of an exemplary central nurse station/multimodality patient-care device/remote patient watch device according to one embodiment of the present disclosure;

FIGS. 5A-5C illustrate examples of GUIs for displaying monitored physiological parameters of a patient and customizable parameter configurations according to embodiments of the present disclosure; and

FIGS. 6A-6C illustrate examples of GUIs for displaying monitored physiological parameters of a patient and customizable parameter configurations via gesture control according to embodiments of the present disclosure.

### DETAILED DESCRIPTION

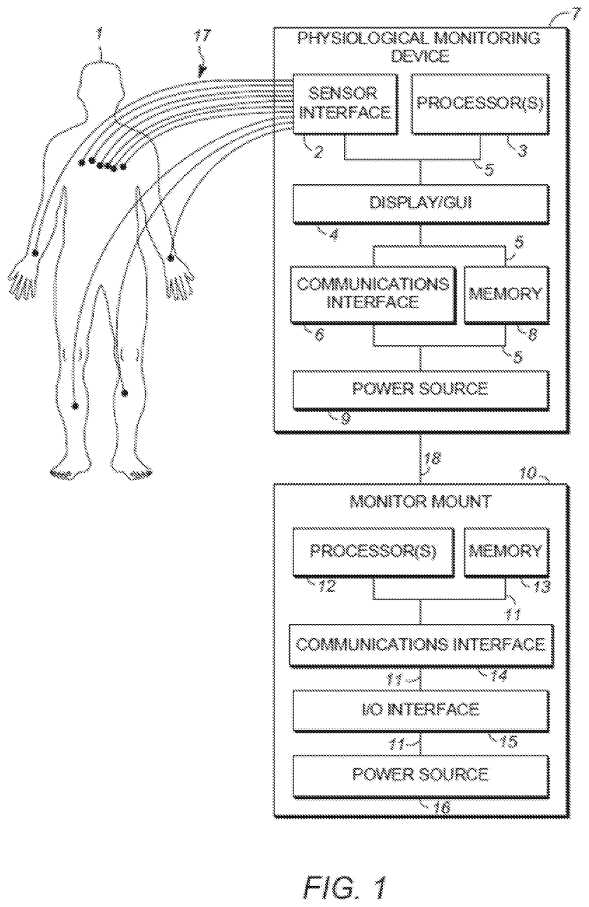

FIG. 1 is a schematic diagram of an exemplary system configured to monitor physiological parameters of a patient and display customizable parameter configurations according to an embodiment of the present disclosure. As illustrated, the system includes a physiological monitoring device 7 configured to receive physiological data from various sensors 17 connected to patient 1, and a monitor mount 10 to which the physiological monitoring device 7 is removably mounted or docked. Physiological monitoring device 7 includes a sensor interface 2, one or more processors 3, a display/GUI 4, a communications interface 6, a memory 8, and a power source 9.

Sensor interface 2 can be implemented in software or hardware and used to connect via wired and/or wireless connections to one or more sensors and/or other medical devices 17 for gathering physiological data from patient 1. The data signals from sensors 17 include, for example, data related to electrocardiogram (ECG), non-invasive peripheral oxygen saturation (SpO₂), non-invasive blood pressure (NIBP), arterial blood pressure (ART) or invasive blood pressure (IBP), heart rate (HR), respiratory rate (RR or Resp), temperature (Temp), ST-segment and/or tidal carbon dioxide (etCO₂), neuromuscular transmission (NMT), cardiac output (CO), apnea detection, and other similar physiological data.

Communications interface 6 allows physiological monitoring device 7 to directly or indirectly (via, e.g., monitor mount 10) to communicate with one or more computing networks and devices. Additionally, communications interface 6 can enable direct (i.e., device-to-device) communications (messaging, signal exchange, etc.) such as from monitor mount 10 to physiological monitoring device 7 using, for example, a USB connection. Communications interface 6 can also enable direct device-to-device connection to other devices such as to a tablet, PC, or similar electronic device; or to an external storage device or memory.

Power source 9 can include a self-contained power source such as a battery pack and/or include an interface to be powered through an electrical outlet (either directly or indirectly via monitor mount 10). Power source 9 can also be a rechargeable battery that can be detached allowing for replacement. In the case of a rechargeable battery, a small built-in backup battery (or supercapacitor) can be provided for continuous power to be provided to physiological monitoring device 7 during battery replacement. Communications between the components of physiological monitoring device 7 (e.g., 2, 3, 4, 6, 8, and 9) are established using an internal bus 5.

Physiological monitoring device 7 is connected to monitor mount 10 via a connection 18 that establishes a communication connection between, for example, the respective communications interfaces 6 and 14 of devices 7 and 10. Connection 18 enables the monitor mount 10 to detachably secure the physiological monitoring device 7 to the monitor mount 10. In this regard, “detachably secure” means that the monitor mount 10 can secure the physiological monitoring device 7, but the physiological monitoring device 7 can be removed or undocked from the monitor mount 10 by a clinical provider when desired.

Monitor mount 10 includes one or more processors 12, a memory 13, a communications interface 14, an I/O interface 15, and a power source 16. One or more processors 12 are used for controlling the general operations of the monitor mount 10. Memory 13 can be used to store any type of instructions associated with algorithms, processes, or operations for controlling the general functions and operations of the monitor mount 10.

Communications interface 14 allows monitor mount 10 to communicate with one or more computing networks and devices (e.g., physiological monitoring device 7). Communications interface 14 can also enable direct (i.e., device-to-device) communications (e.g., messaging, signal exchange, etc.) such as from monitor mount 10 to physiological monitoring device 7 using, for example, a USB connection, coaxial connection, or other similar electrical connection. Communications interface 14 can enable direct (i.e., device-to-device) to other devices such as to a tablet, PC, or similar electronic device; or an external storage device or memory.

I/O interface 15 can be an interface for enabling the transfer of information between monitor mount 10, one or more physiological monitoring devices 7, and external devices such as peripherals connected to monitor mount 10 that need special communication links for interfacing with processor(s) 12.

Power source 16 can include a self-contained power source such as a battery pack and/or include an interface to be powered through an electrical outlet (either directly or by way of physiological monitoring device 7). Power source 16 can also be a rechargeable battery that can be detached allowing for replacement. Communications between the components of monitor mount 10 (e.g., 12, 13, 14, 15, and 16) are established using an internal bus 11.

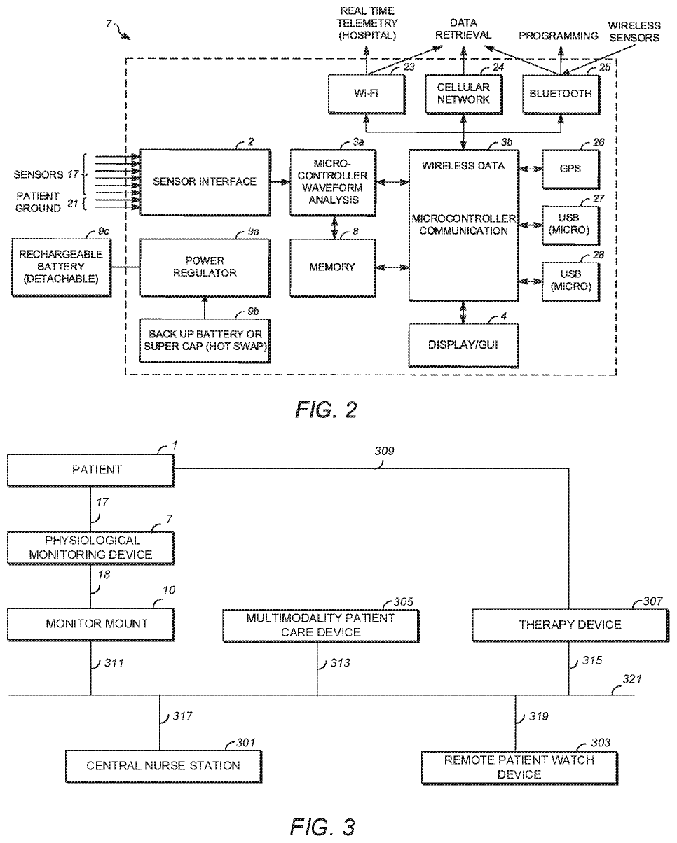

FIG. 2 is a schematic diagram of an exemplary device configured to monitor physiological parameters of a patient and displaying customizable parameter configurations according to an embodiment of the present disclosure. In consistency with FIG. 1, physiological monitoring device 7 is attached to different types of sensors 17 for recording physiological parameters associated with the patient. Physiological monitoring device 7 can also be connected to other wireless sensors and medical devices using communication interface 6 as shown in FIG. 1, which includes circuitry for receiving data from and sending data to one or more devices using, for example, a Bluetooth connection 25. Communications interface 6 in FIG. 1 is represented in FIG. 2 by the combination of microcontroller 3b and elements 23-28.

In consistency with FIG. 1, physiological data collected from sensors 17 are received, via sensor interface 2, and processed by physiological monitoring device 7. Processor(s) 3 shown in FIG. 1 are represented in FIG. 2 as microcontrollers 3a and 3b. Microcontroller 3a, for example, analyzes the digital waveforms to identify certain digital waveform characteristics and threshold levels indicative of conditions (abnormal and normal) of patient 1 using methods known in the art. Microcontroller 3a includes a memory or uses memory 8. The memory stores software or algorithms with executable instructions and microcontroller 3a can execute a set of instructions of the software or algorithms in association with executing different operations and functions of the physiological monitoring device 7, such as analyzing the digital data waveforms related to the data signals from sensors 17. The results of the operations performed by the microcontroller 3a are passed to microcontroller 3b. The microcontroller 3b includes a memory or uses the memory 8.

As noted above, communication interface 6 shown in FIG. 1 is represented by the combination of microcontroller 3b and elements 23-28 in FIG. 2. For example, microcontroller 3b includes communication interface circuitry for establishing communication connections with various devices and networks using both wired and wireless connections, and transmitting physiological data, patient and transport information (e.g., transport times and patient location information), results of the analysis by microcontroller 3a, and alerts and/or alarms to clinical providers. Memory 8 stores software or algorithms with executable instructions and microcontroller 3b can execute a set of instructions of the software or algorithms in association with establishing the communication connections.

As shown in FIG. 2, wireless communication connections established by the communication interface circuitry of microcontroller 3b include a Bluetooth connection 25, a cellular network connection 24, and a Wi-Fi connection 23. The wireless communication connections can allow, for example, patient information (e.g., physiological data and corresponding alerts/alarms, medical record, locations of receiving medical treatment) and hospital information to be transmitted in real-time within a hospital wireless communications network (e.g., Wi-Fi) as well as allow for the information to be transmitted in real-time to other devices (e.g., Bluetooth 25 and/or cellular networks 24).

It is also contemplated by the present disclosure that the communication connections established by microcontroller 3b enable communications over other types of wireless networks using alternate hospital wireless communications such as wireless medical telemetry service (WMTS), which can operate at specified frequencies (e.g., 1.4 GHZ). Other wireless communication connections can include wireless connections that operate in accordance with, but are not limited to, IEEE802.11 protocol, a Radio Frequency For Consumer Electronics (RF4CE) protocol, ZigBee protocol, and/or IEEE802.15.4 protocol.

Bluetooth connection 25 can also be used to provide the transfer of data to a nearby device (e.g. tablet) for review of data and/or changing of operational settings of the physiological monitoring device 7. The microcontroller 3b of the physiological monitoring device 7 provides a communication connection by direct wired (e.g., hard-wired) connections for transferring data using, for example, a USB connection 27 to a tablet, PC, or similar electronic device; or using, for example, a USB connection 28 to an external storage device or memory. Additionally, Microcontroller 3b includes a connection to a display 4 including a GUI for displaying patient information, physiological data or measured data, measurement schedules, alerts/alarms for the patient, clinicians, and/or caregiver's information. Although the physiological monitoring device 7 is described in FIG. 1 as having two microcontrollers 3a and 3b, it is contemplated by the disclosure of the present application that one microcontroller can be implemented to perform the functions of the two microcontrollers 3a and 3b.

As shown in FIG. 2, the physiological monitoring device 7 includes a global positioning system (GPS) or other location data system 26 that can be connected to the communication interface circuitry of microcontroller 3b so that the physiological monitoring device can transmit to the clinician, caregiver, or other devices the location of the patient 1 at all times including the location of the patient 1. Additionally, the location of patient 1 can be used by the microcontroller 3b to determine an estimated time of arrival of patient 1.

For example, location data provided by location data system 26, which may include information on a floor level, can be compared to stored information related to a hospital layout or a hospital map as well as information related to a patient's scheduled care (e.g., treatment or procedure scheduled for the patient 1 in a patient care area within the hospital). Based on the comparison results, microcontroller 3b can determine the estimated time of arrival of patient 1 to the patient care area within the hospital. The estimated time of arrival can be transmitted by the communication interface circuitry of microcontroller 3b to, for example, the hospital wireless communications system.

Additionally, if it is determined by microcontroller 3b that patient 1 is not within the vicinity of the hospital wireless communications system (e.g., based on input from the location data system 26), the pertinent physiological data can be recorded and stored in the memory 8. Additionally, if Bluetooth connection 25 or WIFI connection 23 are not available (e.g., out of transmission range or not operable), then the microcontroller can store the physiological data in the memory 8 for later transmission when the Bluetooth connection or WIFI connection becomes available.

Power source 9 shown in FIG. 1 is represented by elements 9a-9c in FIG. 2. As shown in FIG. 2, the power can be supplied using a rechargeable battery 9c that can be detached allowing for replacement. Rechargeable battery 9c is, for example, a rechargeable lithium-ion battery. Additionally, a small built-in backup battery 9b (or supercapacitor) is provided for continuous power to the physiological monitoring device 7 during battery replacement. A power regulator or regulation circuit 9a is provided between rechargeable battery 9c and small backup battery 9b to control which battery provides power to physiological monitoring device 7. Physiological monitoring device 7 also includes a patient ground connection 21, which can be used as a ground for single-ended unipolar input amplifiers (e.g., precordial leads), or as a ground for bipolar input amplifiers (e.g., limb leads). It is also contemplated by the present disclosure that power regulator 9a can include a self-contained power source such as a battery pack and/or include an interface to be powered through an electrical outlet (either directly or by way of monitor mount 10). Communication between the components of physiological monitoring device 7 can be established using an internal bus similar to internal bus 5 discussed with reference to FIG. 1.

FIG. 3 is a schematic diagram of an exemplary system including a network, a physiological monitoring device, an expanded display, which is referred to as multimodality patient-care device hence forth, a remote patient watch device, a central nurse station and a therapy device. FIG. 3 includes patient 1 who is under monitoring and therapy procedures, physiological monitoring device 7, and the monitor mount 10 that were already discussed with reference to FIGS. 1 and 2. FIG. 3 also includes the addition of a central nurse station 301, a remote patient watch device 303, a multimodality patient-care device 305 and a therapy device 307.

Physiological monitoring device 7 can be connected to network 321 via connection 311 using the communication interface circuitry of communications interface 14 of physiological monitor mount 10 described in FIG. 1. When physiological monitoring device 7 is undocked from monitor mount 10, it can be connected to network 321 via wireless connection using the communication interface circuitry of communications interface 6. Network 321 can be a hospital network.

In one embodiment, physiological monitoring device 7 may transmit, via connection 311, physiological data collected by the sensors, parameter configurations and/or other patient information (e.g., measurement schedules, patient location information, alert/alarm information) to the network 321 for storage and data processing. For example, upon the measurement of physiological parameters using physiological monitoring device 7, the measurement data along with related information may be transmitted by the monitoring device 7 and stored in other devices that are connected to network 321, including central nurse station 301, remote patient watch device 303, multimodality patient care device 305, therapy device 307, etc. Additionally, physiological monitoring device 7 may store configurations (including default configurations and saved configuration settings by clinical providers), or receive configurations received from network 321. Thus, clinical providers are allowed to the quickly use default or previously stored configurations as needed. Physiological monitoring device 7 may also transmit physiological parameter data and configurations via network to multimodality patient-care device 305 for expanded physiological data visualization and also to remote patient watch device 303. Thus, while being away from the patient bed, a clinical provider is able to have a real-time view of physiological parameters with the same configurations as shown on the bedside physiological monitoring device.

Devices including central nurse station 301, remote patient watch device 303, and multimodality patient care device 305 may transmit control signals, via network 321, to control the functions of physiological monitoring device 7 and the sensors that are connected to the device. As such, clinical providers are allowed to control the physiological measurements performed by the sensors or configure the measurement settings while being away from patient bed. For example, remote patient watch device 303 and multimodality patient care device 305 may allow clinical providers to configure display settings (e.g., customize the priority of parameters and corresponding waveforms to be displayed) without being in front of physiological monitoring device 7.

Optionally or additionally, central nurse station 301, remote patient watch device 303 and multimodality patient care device 305 may store the patient's physiological measurements and algorithms to provide recommended configurations to clinical providers. For example, based on the patient's medical conditions, medical history, and/or the care area where the patient is located, the algorithms stored in central nurse station 301, remote patient watch device 303 and multimodality patient care device 305 may provide recommended configurations in display settings by prioritizing the display of specific physiological parameters that are most critical or relevant. For example, some key parameters like SpO₂ and HR may have higher priority than other parameters. Additionally, the parameters that have been reporting alarms most recently may have higher priority than other parameters that have been stable without recent alarms.

As illustrated in FIG. 3, physiological monitoring device 7 may be connected with a remote patient watch device 303 via network 321. Connected to network 321 via connection 319 (e.g., wireless connection), remote patient watch device 303 receives real-time physiological data from physiological monitoring device 7 and other patient-related information from network 321. It allows clinical providers to review the patient's physiological parameters and customizable parameter configurations synchronized with physiological monitoring device 7. Additionally, remote patient watch device 303 also allows clinical providers to review patient information not acquired by or readily visible from physiological monitoring device 7, including physiological data kept as a relatively lower priority and other patient information related to the patient's therapy, diagnosis, and other medical histories. Thus, when being physically remote from the patient bed, clinical providers are still able to review the patient's physiological parameters in real-time as well as other related clinical information, in order to reach clinical decisions.

As further illustrated in FIG. 3, physiological monitoring device 7 may be connected with a multimodality patient-care device 305 via network 321. Connected to network via connection 313 (e.g., wired or wireless), multimodality patient-care device 305 may receive real-time patient physiological data from physiological monitoring device 7. When the patient is also connected to a therapy device 307 via connection 309, multimodality patient-care device 305 may receive corresponding information from therapy device 307 that is also connected to network 321 via connection 315. In an acute care environment, for example, a patient may be connected to a ventilator and an anesthesia machine, while his physiological parameters are monitored in realtime. Thus, multimodality patient-care device 305 integrates and displays clinical information related to a particular patient from different sources, where the clinical information not only includes real-time physiological parameter data but also therapy-related information, lab results, medical history, and others. The real-time physiological parameter data received from the physiological monitoring device is only displayed within a limited area on the user interface, despite multimodality patient-care device 305 commonly has a larger screen compared to a portable patient monitor.

Similar to remote patient watch device 303 as described above, multimodality patient-care device 305 allows clinical providers to review the patient's physiological parameters and customizable parameter configurations synchronized with physiological monitoring device 7. Additionally, remote patient watch device 303 also allows clinical providers to review patient information not acquired by or readily visible from physiological monitoring device 7, including physiological data kept as a relatively lower priority and other patient information related to the patient's therapy, diagnosis, and other medical histories. Thus, when being physically remote from the patient bed, clinical providers are still able to review the patient's physiological parameters in real-time as well as other related clinical information, in order to reach clinical decisions.

As further illustrated in FIG. 3, central nurse station 301 is connected to network 321 via connection 317. Unlike multimodality patient-care device 305 displaying clinical information regarding a particular patient, central nurse station 301 receives information from a plurality of physiological monitoring devices via network 321. In other words, central nurse station 301 receives and displays real-time physiological data corresponding to multiple patients. The real-time physiological parameter data received from each physiological monitoring device is only displayed within a limited area on the user interface of central nurse station 301, despite it has a larger screen compared to a portable patient monitor. For example, central nurse station may display as many as sixteen patients' real-time physiological parameter data simultaneously and thus, it is critical for clinical providers to easily navigate parameter configurations displayed on the central nurse station based on the priorities in clinical conditions of different patients and for each patient, priorities in different physiological parameters.

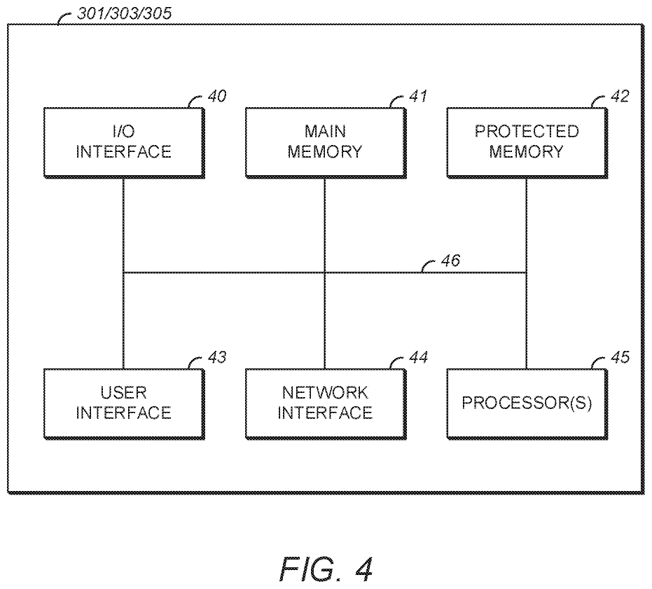

FIG. 4 is a schematic diagram of an exemplary central nurse station/multimodality patient-care device/remote patient watch device according to one embodiment of the present disclosure. As shown in FIG. 4, the exemplary central nurse station 301, remote patient watch device 303 or multimodality patient-care device 305 includes an I/O interface 40, a main memory 41, a protected memory 42, a user interface 43, a network interface 44, and processor(s) 45.

I/O interface 40 can be implemented to accommodate various connections to central nurse station 301, remote patient watch device 303, or multimodality patient-care device 305. I/O interface 40 can be an interface for enabling the transfer of information between 301/303/305 and physiological monitoring device(s) 7 as well as external devices such as peripherals connected to 301/303/305 that need special communication links for interfacing with processor(s) 45. Main memory 41 can be used to store any type of instructions associated with algorithms, processes, or operations for controlling the general functions of 301/303/305 as well as any operating system such as Linux, UNIX, Windows server, or other customized and proprietary operating systems. Protected memory 42 is, for example, a processor reserved memory of dynamic random-access memory (DRAM) or other reserved memory module or secure memory location for storing more critical information such as confidential or proprietary patient information. User interface 43 is implemented for allowing communication between a clinical provider with 301/303/305. The network interface 44 is a software and/or hardware interface implemented to establish a connection between the central nurse station 301 and physiological monitoring device(s) or other servers/central computer inside and outside the patient care or hospital environment. Processor(s) 45 are used for controlling the general operations of central nurse station 301, remote patient watch device 303 or multimodality patient-care device 305. Communication between the components within the central nurse station 301, remote patient watch device 303, or multimodality patient-care device 305 (e.g., 40-44) are established using an internal bus 46.

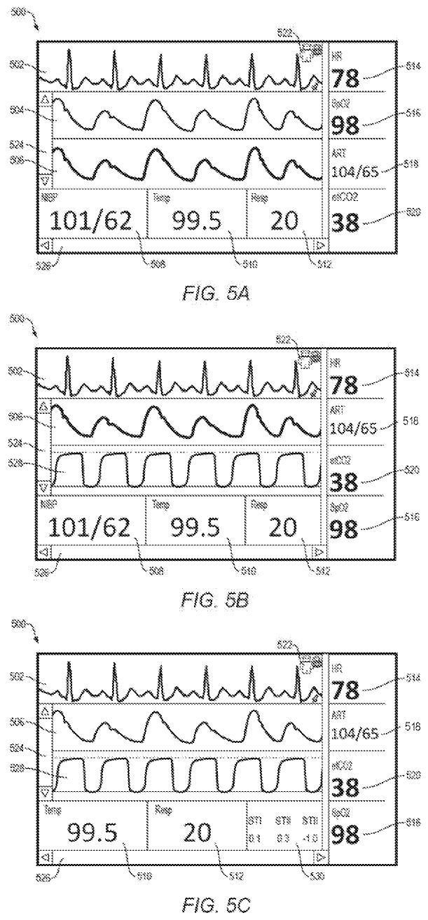

FIGS. 5A-5C illustrate examples of GUIs for displaying monitored physiological parameters of a patient and customizable parameter configurations according to embodiments of the present disclosure. As illustrated in FIG. 5A, display area 500 on the user interface of an electronic device that displays real-time physiological parameter monitoring data (e.g., at least one of devices 7, 301, 303, and 305 in FIGS. 1-4) includes a plurality of sub-areas displaying parameter waveforms and measurements in numerical forms. For example, sub-areas 502, 504, and 506 display real-time waveforms for the measured parameters HR, SpO₂, and ART, respectively. Correspondingly, sub-areas 514, 516, 518 and 520 display numerical values of the measured parameters HR, SpO₂, ART and etCO₂, respectively. Additionally, sub-areas 508, 510, and 512 display numerical values of other parameters including NIBP, temperature (Temp), and raspatory rate (Resp), respectively, where their corresponding waveforms may not be apparent on the GUI.

In one embodiment, one or more sub-areas may be locked. For example, as illustrated in FIG. 5A, sub-areas 502 and 514 displaying the waveform and numerical value of the measured parameter HR may be locked. Optionally, one or more visual indicators 522 may be displayed indicating the locking status of the corresponding parameter. One or more sub-areas may be scrollable, for example, sub-areas 504 and 506 displaying the waveforms of the measured parameters SpO₂ and ART are scrollable. The sub-areas displaying numerical values of the measured parameters may be scrollable, for example, sub-areas 508, 510, and 512 illustrated in FIG. 5A.

Clinical providers are allowed to navigate the scrollable sub-areas to display a variety of parameters. For example, scrollable sub-areas may include a visual indicator indicating the scrollable status of the sub-area (e.g., scroll bars 524 and 526). Upon user's navigation via the scroll bar, different parameters that are not readily apparent can be displayed, for example, the waveform of etCO₂ is displayed in sub-area 528 as illustrated in FIG. 5B. Similarly, clinical providers are allowed to navigate the sub-areas for displaying parameters in numerical values, for example, numerical values of measured ST segment are displayed in sub-area 530 as illustrated in FIG. 5C. Thus, a variety of parameters can be configured to be displayed on the GUI of the patient monitor device with a small-sized screen, clinical providers are allowed to freely navigate and view different parameters as needed without switching among multiple menus, and therefore, reach clinical decisions in an efficient and accurate manner. On the other hand, one or more parameters that are clinically critical are configured to be locked and always apparent on the display while clinical providers navigate other parameters in the scrollable display area.

The configurations for the types of parameters displayed in locked and scrollable sub-areas, respectively, can be automatically initiated by the patient monitor device. Based on the clinical significance of the parameters, the patient monitor device may display a default configuration by, for example, displaying SpO₂ and/or HR in the locked sub-areas (waveform or numerical value), and all other parameters in the scrollable sub-areas. Alternatively, based on the measured parameters received from the plurality of sensors, and other patient-related information (patient location, medical treatment thereunder, medical history, etc.), the patient monitor device may analyze the clinical significance and relevance of different parameters and display the parameters that are clinically significant and/or relevant in the locked sub-areas and other parameters in the scrollable sub-areas. For example, the patient monitor device may automatically display in the locked sub-area a parameter that generated an alarm most recently.

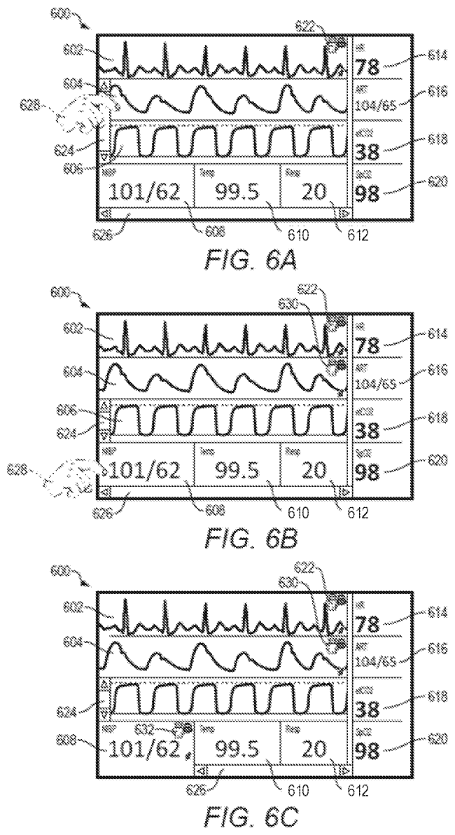

The present disclosure provides additional flexibility to allow clinical providers to manually configure the numbers and types of parameters in the locked and scrollable sub-areas, respectively. FIGS. 6A-6C illustrate examples of GUIs for displaying monitored physiological parameters of a patient and customizable parameter configurations via gesture control according to embodiments of the present disclosure. The gesture control may include but not limited to tap, drag-and-drop, touchdown-and-drag, touchdown-and-hold, flick, pinch, spread, swipe, or any combination thereof.

Similar to FIGS. 5A-5C, display area 600 in FIGS. 6A-6C on the user interface of an electronic device that displays real-time physiological parameter monitoring data (e.g., at least one of devices 7, 301, 303, and 305 in FIGS. 1-4) includes a plurality of sub-areas displaying parameter waveforms and measurements in numerical forms. For example, sub-areas 602, 604, and 606 display real-time waveforms for the measured parameters HR, ART and etCO₂, respectively. Correspondingly, sub-areas 614, 616, 618 and 620 display numerical values of the measured parameters HR, ART, etCO₂ and SpO₂ respectively. Additionally, sub-areas 608, 610, and 612 display numerical values of other parameters including NIBP, Temp), and Resp, respectively, where their corresponding waveforms may not be apparent on the GUI.

As further illustrated in FIGS. 6A-6B, upon the user's gesture control (see 628), the locked area is expanded to include both sub-areas 602 and 604, each including a visual indicator (622 and 630) indicating the locking status of the corresponding parameter. Correspondingly, the scrollable area is limited to sub-area 606 displaying real-time waveforms of etCO₂, as well as other parameters that will be displayed upon clinical providers' navigation using scroll bar 624. As further illustrated in FIGS. 6B-6C, clinical providers are also allowed to move the numerical value for the parameter NIBP displayed in scrollable sub-area 608 to a locked sub-area by gesture control, where a visual indicator 632 is displayed indicating the locking status of the corresponding parameter. Correspondingly, the scrollable area is limited to sub-areas 610 and 612 displaying numerical values of Temp and Rasp, as well as other parameters that will be displayed upon clinical providers' navigation using scroll bar 626. Similarly, the patient monitor device provides the flexibility to allow clinical providers to move the waveform or numerical value of a parameter displayed in a locked sub-area to a scrollable sub-area, via gesture control. When the parameter is moved from the locked sub-area to the scrollable sub-area, the visual indicator showing the locking status is removed or replaced by a visual indicator indicating its scrollable status.

Advantageously, the present disclosure allows a variety of parameters and other patient-related information to be displayed on the GUI of a patient monitoring device with a small-sized display (e.g., physiological monitoring device 7 as illustrated in FIGS. 1-3 and remote patient watch device 303 as illustrated in FIGS. 3-4) or an electronic device that only allows a small portion of its GUI to display each patient's real-time physiological parameter data (e.g., central nurse station 301 and multimodality patient-care device 305 as illustrated in FIGS. 3-4). By providing locked and scrollable sub-areas on the GUI, it allows clinical providers to adjust the types and forms of the displayed parameters in the locked and scrollable sub-areas, based on the clinical priority of the parameters, and/or users' needs.

For example, the present disclosure allows patient-related information, including realtime physiological parameters of the patient to be displayed on a central nurse station in a customizable manner. Commonly, a central nurse station displays no more than sixteen patients' information simultaneously on the GUI (i.e., cluster view), with each patient's information compacted in a limited display area. The present disclosure allows customizable configurations for clinical providers to navigate the visualization of physiological parameters and other patient-related information, based on the priorities in parameters and clinical conditions of each patient. Additionally, the present disclosure allows more patients' clinical information to be displayed by locking the physiological parameters and other information corresponding to patients with higher clinical priority. For other patients who are either new patients and not yet assigned to the central station cluster view or deemed as lower clinical priority, their information will be displayed in a scrollable area. Thus, the present disclosure provides a dynamic view of more patients' information, such that clinical providers can monitor patients with higher priority as their information is apparent and easily navigate other patients' information displayed within the scrollable area as needed.

It should be noted that the aforementioned embodiments are for exemplary purposes only. The present disclosure does not intend to limit the location and arrangement of the locked and/or scrollable sub-areas on the GUI. Furthermore, the present disclosure does not intend to limit the types of parameters displayed in waveform and/or numerical values.

It is contemplated that the implementation of the components of the present disclosure can be done with any newly arising technology that may replace any of the above implementation technologies.

In general, it is contemplated by the present disclosure that the electronic devices (e.g., device 7 and device mount 10 as illustrated in FIGS. 1-2, device 7, 301, 303, and 305 as illustrated in FIGS. 3-4) include electronic components or electronic computing devices operable to receive, transmit, process, store, and/or manage patient data and information associated performing the functions of the system, which encompasses any suitable processing device adapted to perform computing tasks consistent with the execution of computer-readable instructions stored in a memory or a computer-readable recording medium. Further, any, all, or some of the computing devices in the electronic devices (e.g., device 7 and device mount 10 as illustrated in FIGS. 1-2, device 7, 301, 303,305 as illustrated in FIGS. 3-4) may be adapted to execute any operating system, including Linux, UNIX, Windows Server, etc., as well as virtual machines adapted to virtualize execution of a particular operating system, including customized and proprietary operating systems. The physiological monitoring device and the monitor mount are further equipped with components to facilitate communication with other computing devices over one or more network connections, which may include connections to local and wide area networks, wireless and wired networks, public and private networks, and any other communication network enabling communication in the system.

Hardware processors described in the present disclosure (e.g., processor(s) 3 and 12 as illustrated in FIG. 1, microprocessor(s) 3a and 3b as illustrated in FIG. 2, and processor(s) 45 in FIG. 4) are used for controlling the general operations of a patient monitoring device (e.g., devices 7, 10 as illustrated in FIGS. 1-2, and devices 301, 303, and 305 as illustrated in FIGS. 3-4). Each one of the one or more processors can be, but are not limited to, a central processing unit (CPU), a hardware microprocessor, a multi-core processor, a single-core processor, a field-programmable gate array (FPGA), a microcontroller, an application-specific integrated circuit (ASIC), a digital signal processor (DSP), or other similar processing devices capable of executing any type of instructions, algorithms, or software for controlling the operation and performing the functions of the physiological monitoring device. Hardware processors described in the present disclosure may also be configured to execute modules by software, hardware, firmware, or any combinations thereof, and other mechanisms for configuring processing capabilities on the processors. As described in the present disclosure, “module” may refer to any component or set of components that perform the functionality attributed to the module. It may include one or more physical processors during execution of processor-readable instructions, circuitry, hardware, storage media, and/or any other components.

Memory described in the present disclosure (e.g., memory 8 and 13 as illustrated in FIGS. 1-2, and memory 41 and 42 as illustrated in FIG. 4) can be a single memory or one or more memories or memory locations that include, but are not limited to, a random access memory (RAM), a memory buffer, a hard drive, a database, an erasable programmable read only memory (EPROM), an electrically erasable programmable read only memory (EEPROM), a read only memory (ROM), a flash memory, hard disk or any other various layers of memory hierarchy. The memory can be used to store any type of instructions and patient data associated with algorithms, processes, or operations for controlling the general functions and operations of the physiological monitoring device.

Additionally, the device (e.g., device 7 illustrated in FIGS. 1-2, devices 7, 301, 303, and 305 as illustrated in FIGS. 3-4) includes a display/GUI (e.g., display/GUI 4 illustrated in FIG. 1 and user interface 43 illustrated in FIG. 4). The display/GUI is for displaying various patient data and hospital or patient-care information and includes a user interface implemented for allowing communication between a clinical provider and the electronic device. The display/GUI includes, but is not limited to, a keyboard, a liquid crystal display (LCD), cathode ray tube (CRT), thin-film transistor (TFT), light-emitting diode (LED), high definition (HD), or other similar display devices with touch screen capabilities. The patient information displayed can, for example, relate to the measured physiological parameters of the patient as well as information related to the transporting of the patient. The size of the display 4 as illustrated in FIGS. 1 and 2 may vary in a range of 5 inches to 12 inches. In some conditions, a small-sized physiological monitoring device 7 (e.g., 7 inches) may be coupled to a larger-sized display (e.g., 15 inches). Furthermore, the size of the display on a multimodality patient-care device 305 as illustrated in FIG. 3 may vary from 20 inches to 25 inches, as it displays not only real-time monitoring information received from physiological monitoring device 7, but also other clinical information corresponding to the patient from one or more therapy devices 307 and other devices. Additionally, remote patient watch device 303 as illustrated in FIG. 3 may include a mobile phone, a tablet and thus, the size of the display may vary from 5 inches to 13 inches.

Communications interfaces 6 and 14 as illustrated in FIG. 1 can include various network cards, interfaces, or circuitry to enable wired and wireless communications with such computing networks and devices. The communications interface 6 can also be used to implement, for example, a Bluetooth connection, a cellular network connection, and/or a WiFi connection. Other wireless communication connections implemented using communications interface 6 include wireless connections that operate in accordance with, but are not limited to, IEEE802.11 protocol, a Radio Frequency for Consumer Electronics (RF4CE) protocol, ZigBee protocol, and/or IEEE802.15.4 protocol.

Network interface 44 as illustrated in FIG. 4 includes software and/or hardware interface circuitry for establishing communication connections with the rest of the system using both wired and wireless connections for establishing connections to, for example, a local area networks (LANs), wide area networks (WANs), metropolitan area networks (MANs) personal area networks (PANs), and wireless local area networks (WLANs), system area networks (SANS), and other similar networks.

I/O interface 15, connection 18 as illustrated in FIG. 1 and I/O Interface 40 as illustrated in FIG. 4 can be implemented to accommodate various connections to the monitor mount 10 that include but is not limited to, a universal serial bus (USB) connection, parallel connection, a serial connection, coaxial connection, a High-Definition Multimedia Interface (HDMI) connection, or other known connection in the art connecting to external devices.

A computer-readable medium can comprise DRAM, RAM, ROM, EEPROM, CD-ROM or other optical disk storage, magnetic disk storage or other magnetic storage devices, or any other medium that can be used to carry or store desired computer-readable program code in the form of instructions or data structures and that can be accessed by a general-purpose or special-purpose computer, or a general-purpose or special-purpose processor. Disk or disc, as used herein, include compact disc (CD), laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The detailed description is made with reference to the accompanying drawings and is provided to assist in a comprehensive understanding of various example embodiments of the present disclosure. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments. In addition, descriptions of well-known functions and constructions may be omitted for clarity and conciseness. Accordingly, those of ordinary skill in the art will recognize that various changes and modifications of the examples described herein can be made without departing from the spirit and scope of the present disclosure.

Use of the phrases “capable of,” “capable to,” “operable to,” or “configured to” in one or more embodiments, refers to some apparatus, logic, hardware, and/or element designed in such a way to enable the use of the apparatus, logic, hardware, and/or element in a specified manner. The subject matter of the present disclosure is provided as examples of apparatus, systems, methods, circuits, and programs for performing the features described in the present disclosure. However, further features or variations are contemplated in addition to the features described above. It is contemplated that the implementation of the components and functions of the present disclosure can be done with any newly arising technology that may replace any of the above-implemented technologies.

Various modifications to the disclosure will be readily apparent to those skilled in the art, and the generic principles defined herein may be applied to other variations without departing from the spirit or scope of the present disclosure. Throughout the present disclosure the terms “example,” “examples,” or “exemplary” indicate examples or instances and do not imply or require any preference for the noted examples. Thus, the present disclosure is not to be limited to the examples and designs described herein but is to be accorded the widest scope consistent with the principles and novel features disclosed.

1. An electronic device capable of executing a customizable configuration for displaying one or more physiological parameters of a patient comprising:
a display configured to display the one or more physiological parameters of the patient; and
one or more processors configured to:
provide a graphical user interface (GUI) on the display, the GUI including:
a first sub-area configured to display a measured value of the one or more physiological parameters, and a visual indicator indicating a locking status of the first sub-area, and
a second sub-area configured to display a measured value of at least two of the one or more physiological parameters, and a visual indicator indicating a scrollable status of the second sub-area, and
upon receipt of an input, cause the measured value of the one or more physiological parameters to be displayed in one of the first sub-area and the second sub-area.

2. The electronic device of claim 1, wherein the one or more physiological parameters of the patient comprises electrocardiogram, non-invasive peripheral oxygen saturation, non-invasive blood pressure, arterial blood pressure, invasive blood pressure, heart rate, respiratory rate, temperature, ST segment, tidal carbon dioxide, neuromuscular transmission, cardiac output and apnea.

3. The electronic device of claim 1, wherein the measured value of the one or more physiological parameters is displayed at least in a form of:
a waveform indicating a plurality of measured values over a pre-determined time period corresponding to the one or more physiological parameters; or
a numerical value of the one or more physiological parameters.

4. The electronic device of claim 1, wherein:
upon the receipt of the input, the one or more processors are configured to:
move the measured value of the one or more physiological parameters displayed in the first sub-area to the second sub-area; or
move the measured value of the one or more physiological displayed in the second sub-area to the first sub-area.

5. The electronic device of claim 1, wherein:
the input is provided via a user's gesture control.

6. The electronic device of claim 5, wherein:
the gesture control is in a form of tap, drag-and-drop, touchdown-and-drag, touchdown-and-hold, flick, pinch, spread, swipe, or a combination thereof.

7. The electronic device of claim 1, wherein the one or more processors are further configured to provide at least one of:
a default configuration automatically provided by the electronic device;
a user-determined configuration saved in the electronic device or transmitted via a communication interface of the electronic device, wherein the communication interface is configured to be connected to a communication network; or
an automatically adjusted configuration based on a real-time change in the measured value of the one or more physiological parameters.

8. The electronic device of claim 1, wherein the electronic device is a physiological patient monitor.

9. The electronic device of claim 1, wherein the electronic device is a multimodality patient-care device.

10. The electronic device of claim 1, wherein the electronic device is a remote patient watch device.

11. A method of executing a customizable configuration for displaying one or more physiological parameters of a patient on an electronic device comprising the steps of:
providing a graphical user interface (GUI) on a display of the electronic device, the GUI comprising:
a first sub-area configured to display a measured value of the one or more physiological parameters, and a visual indicator indicating a locking status of the first sub-area, and
a second sub-area configured to display a measured value of at least two of the one or more physiological parameters, and a visual indicator indicating a scrollable status of the second sub-area, and
upon receipt of an input, causing the measured value of the one or more physiological parameters to be displayed in one of the first sub-area and the second sub-area.

12. The method of claim 11, wherein the one or more physiological parameters of the patient comprise electrocardiogram, non-invasive peripheral oxygen saturation, non-invasive blood pressure, arterial blood pressure, invasive blood pressure, heart rate, respiratory rate, temperature, ST segment, tidal carbon dioxide, neuromuscular transmission, cardiac output and apnea.

13. The method of claim 11, wherein the measured value of the one or more physiological parameters is displayed at least in a form of:
a waveform indicating a plurality of measured values over a pre-determined time period corresponding to the one or more physiological parameters; or
a numerical value of the one or more physiological parameters.

14. The method of claim 11, wherein the step of causing the measured value of the one or more physiological parameters to be displayed in one of the first sub-area and the second sub-area further comprising at least one of:
moving the measured value of the one or more physiological parameters displayed in the first sub-area to the second sub-area; or
moving the measured value of the one or more physiological parameters displayed in the second sub-area to the first sub-area.

15. The method of claim 11, wherein:
the input is provided via a user's gesture control.

16. The method of claim 15, wherein:
the gesture control is in a form of tap, drag-and-drop, touchdown-and-drag, touchdown-and-hold, flick, pinch, spread, swipe, or any combination thereof.

17. The method of claim 11, further comprising providing at least one of the following configurations including:
a default configuration automatically provided by the electronic device;
a user-determined configuration saved in the electronic device or transmitted via a communication interface of the electronic device, wherein the communication interface is configured to be connected to a communication network; and
an automatically adjusted configuration based on a real-time change in the measured value of the one or more physiological parameters.

18. The method of claim 11, wherein the electronic device is a physiological patient monitor.

19. The electronic device of claim 11, wherein the electronic device is a multimodality patient-care device.

20. The method of claim 11, wherein the electronic device is a remote patient watch device.
